(19) United States
(12) Patent Application Publication
(10) Pub. No.: US 2009/0099652 A1
(43) Pub. Date: Apr. 16, 2009

(54) Composite Vascular Prosthesis

(75) Inventors: Juan Granada, Pearland, TX (US); Simon M. Furnish, New York, NY (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 12/212,474

(22) Filed: Sep. 17, 2008

Related U.S. Application Data
(63) Continuation of application No. 11/726,986, filed on Mar. 24, 2007, now abandoned.
(60) Provisional application No. 60/785,579, filed on Mar. 24, 2006.

(51) Int. Cl. A61F 2/06

(52) U.S. Cl. 623/1.46; 623/1.15; 623/1.17

(57) ABSTRACT

A novel treatment for atherosclerotic vascular disease is described utilizing the implantation of a thin, conformable biocompatible prosthesis constructed from a composite of various structural and therapeutic scaffolds in combination with one or more bioactive agents. This prosthesis can be delivered into position over a lesion in order to passivate atherosclerotic plaques with minimal remodeling of the artery, or alternatively can be applied with a balloon to passivate the remodeled site. The composite prosthesis itself provides mild structural reinforcement of the vessel wall and an evenly distributed platform for the introduction of bioactive therapeutic agents.

This application claims the benefit of U.S. provisional patent application Ser. Nos. 60/785,579 filed Mar. 24, 2006 and 60/582,643 filed Oct. 19, 2006, each of which is incorporated by reference herein in its entirety.

### FIELD OF THE INVENTION

The invention relates generally to a composite vascular prosthesis and more particularly to a highly conformable and biologically active endovascular system for treating vascular disease by promoting the regeneration of vascular tissue after implantation of the prosthesis.

### BACKGROUND OF INVENTION

The field of percutaneous vascular intervention has been exclusively focused in the treatment of obstructive and symptomatic obstructive vascular disease. In fact, endovascular therapy is exclusively reserved for the patient presenting with symptoms related to an obstruction of the lumen of the vessel. In its simplest form, balloon angioplasty treats vascular obstructions by applying high dilatation forces that split the vessel wall structure resulting in vessel recoil, abrupt closure and high restenosis rates. As a result, metallic vascular scaffoldings are currently used to maintain the acute results achieved after balloon dilatation. These metallic structures are deployed using balloon delivery systems that deliver the device at higher deployment pressures disrupting at different levels the integrity of the elastic structures of the vessel wall architecture. As a consequence of the degree of vascular injury, the vessel reacts by eliciting an exaggerated healing response leading to the formation of abnormal scar tissue or restenosis. In order to prevent the occurrence of exaggerated scar tissue formation, drug-eluting stents deliver anti-proliferative agents by incorporating such medications in a polymeric surface on the surface of the stent. Although effective in reducing the accumulation of scar tissue, current evidence suggest that hypersensitivity and allergic reaction to the polymer retained into the vessel wall occurs after drug eluting stent implantation and that this biological effect may be associated to lethal late thrombotic events. In summary, as shown in FIG. 1, balloon angioplasty is associated with uncontrolled injury, split media and intimal disruption, use of bare metal stents is associated with uncontrolled injury, EEL disruption and vessel overexpansion, and use of drug eluting stents is associated with not only the problems of bare metal stents but also issues of residual polymer, delayed healing and vascular hypersensitivity.

Most of the existing vascular scaffoldings constructed today are based on metals. Self-Expanding (SE) stents are typically constructed from nickel-titanium alloys, fabricated either from laser cut and electro-polished tubing or welded wire braids, coils or other wire mesh forms that allow for a small unexpanded profile to reach distal lesions in tortuous vessels which can be deployed and expanded in place when released from a captive sheath. SE stents are not currently used for coronary applications and typically require both pre and post dilatation with an angioplasty balloon. Not only does this require the use of two or more device interventions to achieve the desired outcome, but also the nature of the self-expanding stent allows for continued long-term expansion in the vessel even 7 to 9 months after implantation, resulting in increased vessel injury. The advantages and disadvantages of SE coronary stents are still debated by physicians, but the global market shows that balloon expandable stents are in widespread use and considered the standard in endovascular treatment.

Balloon expandable stents are plastically deformed via high-pressure balloons and sized based on the most normal reference diameter for a particular lumen vessel diameter, not taking into account the structural or biological plaque features of the stenotic site. The balloon expandable coronary stents do not continue to expand after implantation and in some cases require no pre-dilatation. However, if not properly sized, a great number of the balloon expandable stents may remain under-expanded due to the mechanism of implantation of these devices. While typical balloon angioplasty, with or without a stent has shown definite acute improvements to the state of treatment of heart disease, these technologies have not been demonstrated to significantly decrease the frequency of future cardiovascular events or improvement on long-term survival. Angioplasty is a very traumatic process, primarily due to the high strains induced on the vessel wall from both radial expansion and straightening of the curved vessel. In addition, it has been shown that after balloon angioplasty, split of the plaque components and medial layer of the vessel is the most common mechanism involved in the relief of the obstructed site. Stents are now being combined with drugs, radioactive seeds, thermal and cryogenic temperatures to reduce the problem of restenosis, where the natural reaction to the implant causes proliferation of neointimal growth that may further reduce the diameter of a vessel. These provisions are essentially attempts to patch the original damage induced by the original treatment in some cases inducing further vascular injury instead of facilitating the process of vascular healing.

U.S. Publication No. 2002/0004679 discloses drug eluting polymer stents for treating restenosis with topoisomerase inhibitors, and is incorporated herein by reference in its entirety.

U.S. Publication No. 2002/0125799 discloses intravascular stents for the treatment of vulnerable plaque that consist of opposing end ring portions and a central strut portion having a zig-zag configuration that connects with the end portion at apices of the zig-zag structure, and is incorporated herein by reference in its entirety.

U.S. Publication No. 2005/0137678 discloses a low-profile resorbable polymer stent and compositions therefor, and is incorporated herein by reference in its entirety.

U.S. Publication No. 2005/0287184 discloses drug-delivery stent formulations for treating restenosis and vulnerable plaque, and is hereby incorporated by reference herein in its entirety.

New theories are being developed regarding the nature of the genesis of major acute cardiovascular events such as stroke, myocardial infarction and sudden cardiac death. The vulnerable plaque, the vascular lesion thought to be the anatomical substrate responsible for future cardiovascular events is characterized by a lipid rich pool buried within the vessel and separated from the blood flow by a thin fibrous cap as shown in FIG. 2. When ruptured, the lipid is released into the bloodstream and triggers the formation of a clot that can be carried downstream with deadly consequences. Generally, vulnerable plaque rupture or superficial erosion leads to exposure of thrombogenic materials. A healing response may occur resulting in repair or accelerated progression. Alternatively, thrombosis leading to acute vascular events may occur. Such plaques are invisible to the standard diagnostic methods employed in catheter labs across the globe and have generated a technical and clinical hunt for a new standard in both diagnosis and treatment of these plaques.

A new approach to the treatment of diseased vessels is recommended to reinvestigate the foundations of a minimally invasive approach to treating heart disease. While angioplasty is far less invasive when compared to coronary bypass surgery, there is a constant push to find further techniques to limit the damage caused by the basic procedure in order to treat a disease.

There is a current need for therapies able to locally stabilize and reset the biological behavior of these vascular lesions at risk of disruption. Today, current technology carries significant mechanical, technical and biological disadvantages that should be resolved in order to advance local percutaneous therapy as the standard of care.

### SUMMARY OF INVENTION

There remains a need for a conformable biologically active endovascular device for the treatment of vascular disease.

A novel treatment for atherosclerotic vascular disease is described utilizing the implantation of a thin, conformable biocompatible prosthesis constructed from a composite mixture of various structural and therapeutic scaffolds in combination with one or more bioactive agents. This prosthesis can be delivered into position over a lesion in order to stabilize and change the biological behavior of atherosclerotic plaques with minimal remodeling of the artery, or alternatively can be applied with an angioplasty balloon to passivate and remodel the diseased vascular segment. The composite prosthesis provides structural reinforcement of the vessel wall by covering, compressing and remodeling the plaque contents but not imposing significant vascular injury. Also, the biological components of the prosthesis facilitate device incorporation into the vessel wall and promote vascular healing. In addition, this prosthesis may become an evenly distributed platform for the introduction of biologically active therapeutic agents. The resulting biological matrix follows the principles of a) controlled mechanical remodeling by applying pressure that does not exceed the rupture threshold of the elastic components of the lesion (mechanical stabilization), b) regulating the inflammatory nature of the lesion by facilitating the incorporation of the device into the plaque milieu, therefore, re-setting the biological features of these lesions and c) promotion of vascular healing by directing the adhesion of endothelial cells. As summarized in FIG. 3, the principles include in summary mechanical stabilization/reinforcement of the fibrous cap, promotion of vascular healing, regulation of inflammation and cell growth and prevention/inhibition of thrombosis.

The composite vascular prosthesis of the invention may include: a structural matrix or skeleton, a bioadhesive component and a bioactive component, as exemplified in FIG. 4. The proposed sequence of biological events required to achieve vascular healing following device implantation are described. Upon expansion, the resulting biological matrix modifies the structure and morphology of the atherosclerotic plaque. The expanded matrix further provides mechanical support and scaffolding to stabilize the lesion without exceeding the mechanical forces required to rupture the elastic components of the vessel wall. Once the prosthesis is apposed to the vessel wall, the bioadhesive component signals healthy vascular tissue growth and incorporation of the prosthesis to prevent future migration. The bioadhesive component establishes the conditions necessary for the resident vascular cells and proteins to migrate, grow and populate the device as a precursor to the formation of vascular granulation tissue and eventual formation of a thin, healthy neointimal layer. This bioadhesive component adheres the prosthesis to the vessel wall, stabilizing any fissures, ruptures or vulnerable plaque regions and will contain plaque contents from distal dislodgment. Bioactive agents either infused within or coating atop the base matrix may be needed in order to control the immune response, promote the healing process, regenerate the vascular tissue and aid in the incorporation of the biomaterial prosthesis into the local tissue. The bioactive/biomimicry component may be preferentially located in the luminal aspect of the device and allows the adhesion, recruitment and/or homing of cell precursors of the endothelial layer, thus constructing a new healthy arterial segment within the existing segment.

One embodiment of the invention provides a thin tubular biocompatible vascular prosthesis including a base matrix containing a combination of structural biomaterials and bioactive ingredients infused with a cross linker for selective adhesion to the vessel wall upon expansion.

One embodiment of the invention provides a thin tubular biocompatible vascular prosthesis including a base matrix of alternating layers of elastin, collagen and a biocompatible crosslinking adhesive.

One embodiment of the invention provides a luminal prosthesis including a structural component, an elastic component, an adhesive component and a biostability component.

One embodiment of the invention provides a thin tubular biocompatible vascular prosthesis constructed from a base matrix containing a combination of structural biomaterials and bioactive ingredients infused with a cross linker for selective adhesion to the vessel wall upon expansion, and including a scaffolding of metallic alloys, durable or absorbable polymer(s) or other biological materials. The scaffolding may, for example, be an expandable mesh or framework.

One embodiment of the invention provides a radially expandable vascular luminal prosthesis that includes: a structural component; an abluminal adhesive component; and an adluminal endothelialization-promoting component. In one variation, each of the components is an at least substantially distinct layer with, for example, the structural component disposed at least substantially between the other layers.

A related embodiment of the invention provides a radially expandable vascular luminal prosthesis that includes: a structural component; an adhesive abluminal surface; and an endothial cell-promoting adluminal surface.

In one variation of the embodiments of prostheses according to the invention, the prosthesis exerts a radial expansion force in the range of 30 to 750 mm Hg in a radially expanded state. In a related variation, the prosthesis exerts a radial expansion force in the range of 30 to 250 mm Hg in a radially expanded state. The adhesive abluminal component or surface may be conditionally adhesive, for example, requiring light energy to activate or its adhesiveness or adhesion. The adhesive abluminal component or surface may include at least one protein providing adhesiveness of the prosthesis to a blood vessel wall. The adluminal component or surface may include endothelial cell-promoting structural features and/or endothelial cell-promoting molecules

One embodiment of the invention provides a method for passivating vascular diseases that includes the steps of: loading a prosthesis according to the invention onto an expandable delivery system; positioning prosthesis at tissue region to be treated; expanding the prosthesis to contact the tissue; curing/securing the prosthesis into position; and removing the delivery system. The curing/securing step may include crosslinking proteins within the prosthesis matrix to vascular tissue. The curing/securing step may include crosslinking proteins using light energy activated protein crosslinking compounds, for example, by photoactivating naftalimide with light energy at 405±20 nm.

One embodiment of the invention provides an expandable vascular prosthesis that includes: an at least substantially tubular, radially expandable structural component including an abluminal surface and an adluminal surface; and a bioadhesive coating including at least one biomolecule selected from the group consisting of a collagen and an elastin, wherein the bioadhesive coating is disposed on at least part of, such as at least substantially all of, the abluminal surface of the structural component, and wherein the adluminal surface includes surface features having depths in the range of 5 nm to 5 μm and lateral dimensions in the range of 50 nm to 5 microns, said surface features being present on the adluminal surface at a density of 1 to 500 surface features per 10 μm². In one variation, the depth of the surface features is in the range of 5-200 nm for improving durability of the structural component along with endothelial cell migration and adhesion. In one variation, the prosthesis exerts a radial expansion force from 30 to 750 mm Hg in a radially expanded state. In one embodiment, a reduced radial force from 30 to 250 mmHg is utilized to reduce the degree of injury inflicted on the lesion and vessel. At least part of, such as at least substantially all of, the adluminal surface may also be coated with at least one biomolecule, such as fibronectin, for example, to promote endothelialization of the adluminal surface. The bioadhesive coating may include an activatable protein crosslinker. Upon deploying the prosthesis to its expanded state in a blood vessel, the crosslinker may be activated. The prosthesis may be self-expanding. Self-expansion may be imparted by using a self-expanding structural component such as a shape memory metal alloy such as Nitinol or a shape memory polymer, such as polylactic acid.

The invention also provides methods for treating an atherosclerotic lesion in a blood vessel of a patient that include the steps of: locating a site of an atherosclerotic lesion in a blood vessel of a patient; transporting a prosthesis of the invention in an unexpanded state to the site of the atherosclerotic lesion in the blood vessel; and radially expanding the prosthesis at the site of the atherosclerotic lesion so that the prosthesis contacts the blood vessel wall at the site. The atherosclerotic lesion may, for example, be a vulnerable plaque. The atherosclerotic lesion may, for example, be an atherosclerotic lesion/plaque freshly treated by angioplasty, such as balloon angioplasty, stenting, stent-graft placement, atherectomy, brachytherapy or other therapeutic treatment. The atherosclerotic lesion may, for example, be a restenosis resulting from a prior intervention by angioplasty balloon, stenting, stent-graft placement, atherectomy, brachytherapy or other therapeutic treatment.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 illustrates mechanisms of conventional endovascular therapy.

FIG. 2 illustrates a vulnerable plaque atherosclerotic lesion.

FIG. 3 illustrates biological mechanisms of focal vulnerable plaque therapy.

FIG. 4 illustrates an initial phase in the response of a blood vessel to treatment with a prosthesis embodiment of the invention.

FIG. 5 illustrates a next phase in the response of a blood vessel to treatment with a prosthesis embodiment of the invention

FIG. 6 illustrates a next phase in the response of a blood vessel to treatment with a prosthesis embodiment of the invention

FIG. 7 illustrates an embodiment of a composite vascular prosthesis according to the invention.

FIG. 8 illustrates an embodiment of a composite vascular prosthesis according to the invention.

FIG. 9 illustrates an embodiment of a composite vascular prosthesis according to the invention.

FIG. 10 illustrates an embodiment of a composite vascular prosthesis according to the invention.

FIG. 11 illustrates an embodiment of a composite vascular prosthesis according to the invention.

FIG. 12 illustrates the relationship between induced vessel strain, applied vessel force or pressure and lumen diameter.

FIG. 13 illustrates various mechanical stabilization options for treatment of atherosclerotic lesions.

FIG. 14 illustrates a quilting method embodiment for expansion strain-mediated release of drugs or adhesives.

FIG. 15 illustrates various structural surface modification aspects of the prostheses of the invention.

FIG. 16 illustrates a stent design that may serve as a structural component for a composite vascular prosthesis according to the invention.

FIG. 17 illustrates a composite vascular prosthesis embodiment of the invention that consists of three layers, mounted on a low-pressure balloon delivery catheter.

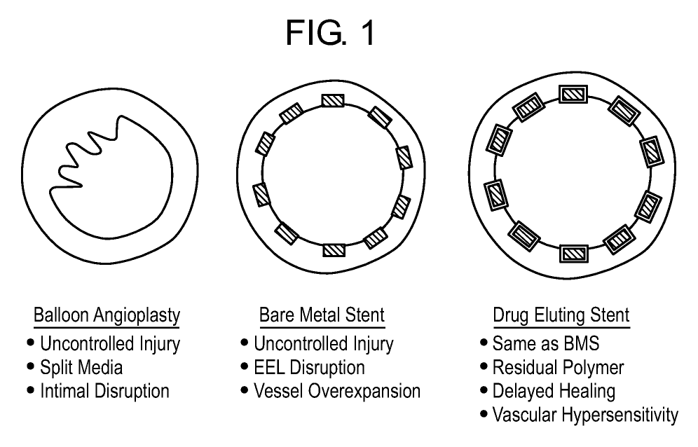

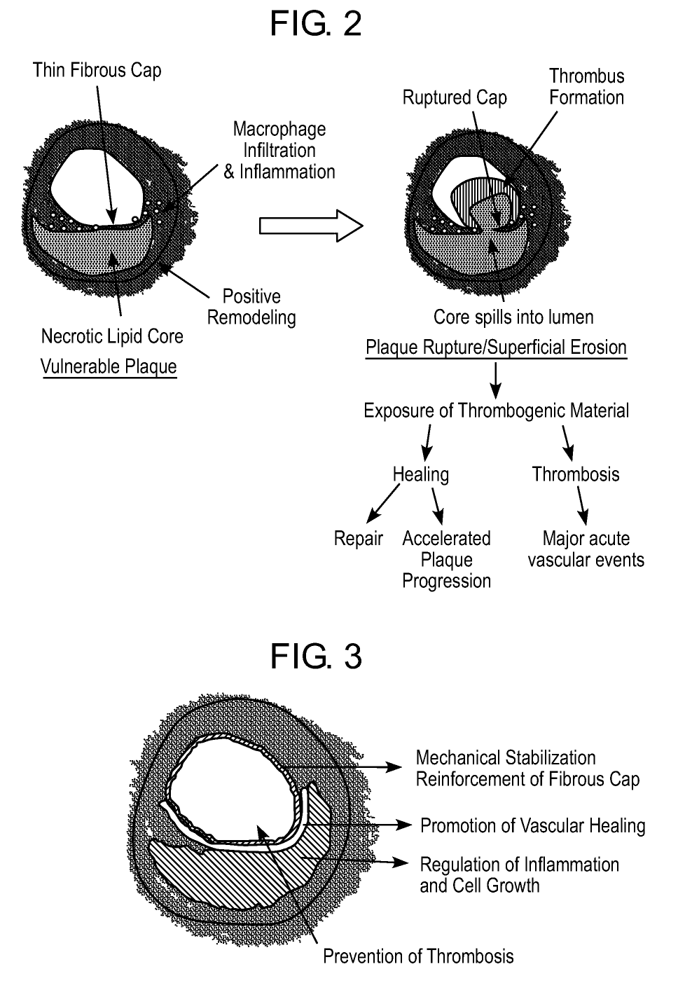

### DETAILED DESCRIPTION OF THE INVENTION

### Overview

Bare metal stents and bioabsorbable stents have relied upon plastic deformation under extreme expansion loads to provide excessive radial support in order to keep their structures in place and maintain patency of the vessel. Unfortunately, such approach results in further injury to a vessel already compromised by disease, which begins to manifest with excessive neointimal growth and restenosis of the vessel. Eluted drugs from a stent decrease the amount of scar tissue formation by suppressing healing. In addition, vascular hypersensitivity reactions and toxic effects have been reported in the literature and seem to be associated to late adverse cardiovascular events. The present invention is an attempt to restore the vessel by minimizing vascular injury imposed by the prosthesis, promoting the growth of healthy tissue and promoting the endothelial coverage of the prosthesis by applying a biologically active surface.

The principles of the invention can also be directed to the passivation of vulnerable plaques (VP) through, for example, (1) structural reinforcement with minimal induced strain on the vessel and (2) regeneration of vascular tissue in-situ through local cell recruitment.

The composite vascular prosthesis in accordance with the principles of the invention can include a multi-layered matrix, a delivery device and an activating process.

### The Composite Vascular Prosthesis:

Structural matrix component. The structural matrix component consists of a skeleton or scaffolding to support the bulk of the mechanical stress imposed by the arterial wall after implantation as a result of lesion and vessel dilation. This component can be included of ultra-thin stainless steel, cobalt chromium alloy, titanium-nickel alloys or other metallic alloys. Additionally, the structural matrix component can be constructed from a combination of one or more synthetic polymers and/or biological materials, such as collagen. The wall thickness of the structural component may, for example, be in the range of 20 to 125 microns, such as in the range of 25 microns to 87 microns, or at or about 0.001 inch to 0.0035 inch. In one embodiment, the wall thickness is 0.0025 inch, or about 62 microns.

Bioadhesive component. The bioadhesive component serves as an anchoring mechanism for attachment to the vessel wall as well as the attachment of various proteins to the structural component. Changing the proportion of these proteins may affect the physical properties of vascular prosthesis in terms of hardness or flexibility. Possible bioadhesive materials include collagen, elastin, hyaluronic acid, chitosin, heparin(s), keratin or other molecules belonging to the extracellular matrix group.

Bioactive component. In order to further reduce the inflammatory response and promote quick natural healing with minimal neointimal growth, a biomimicry component shall be incorporated to the vascular prosthesis. This component is preferentially located in the luminal aspect (interior surface) of the prosthesis but could be applied throughout the entire outer surface of the device. The bioactive component could be part of the structural matrix through modifying its own surface or could be a biological coating that modifies the surface of the whole prosthesis. Possible materials include, fibronectin, vitronectin, laminin, thrombin, fibrinogen, RGD peptides or other ligands that affect endothelial cell adhesion, migration and differentiation.

Delivery Device. There are a variety of ways in which the matrix can be delivered, many of which follow along the well-established techniques of balloon expandable and self-expandable stent delivery systems. Balloon expandable systems utilize a collapsed and folded high-pressure balloon, often constructed from nylon, polyester or other thin polymer. The prosthesis is compressed around the balloon to a low profile (around 1 mm in diameter) for accessing coronary arteries. When the prosthesis is co-located with the targeted lesion, the balloon is inflated, and as it expands it expands the prosthesis into the vessel wall. Self-expanding stent systems utilize a highly compressed prosthesis with built-in expansion which is stuffed within a small sheath. Relative motion between the sheath and a pusher rod extending proximal and adjacent to the prosthesis within the sheath results in incremental release of the prosthesis as it is emerges from beneath the sheath. Hybrid balloon/sheath systems also exist in the prior art and could be adapted to the novel prosthesis described herein. Delivery of the composite vascular prosthesis could further benefit from use of low-trauma delivery systems designed to limit the applied forces and resulting vessel injury due to the expansion forces generated. The catheter-based delivery systems for vascular prostheses provided in U.S. Publication No. 2006/0271154, which is incorporated by reference herein, may also be used.

Activating Process. In the preferred embodiments, the bioadhesive and biomimicry matrix components are integrated into preformed scaffolding. In alternate embodiments the biological material may be an expandable or stretchable structure, which may need additional radial strength to prevent vessel prolapse. One solution is to enhance the inherent adhesive mechanisms present with an applied chemical, energy or strain based activator. In situ cross-linking within the various components of the structural matrix may also be used to increase the scaffolding properties and further prevent negative remodeling once the delivery system (balloon catheter, etc.) has been collapsed and removed. Once the membrane of biological material is expanded, these activating processes—which in various embodiments can include chemical activation based upon release or exposure to a secondary chemical or biochemical catalyst for cross-linking, light-activated cross-linking or in-situ photo-polymerization process, thermal activation (cold or heat), or activation via application of ultrasonic energy. The activation measures may be incorporated into the delivery system, applied though secondary means such as via guidewire or bolus injection through the guide catheter, intravenous injection or a chemical catalyst residing dormant within the base matrix which is exposed and activated upon expansion during deployment.

### DESCRIPTION

A novel prosthesis in accordance with the principles of the invention is described herein. The preferred concept is a thin, flexible tubular composite matrix constructed from biocompatible components that is delivered in a collapsed form and expanded to be placed in contact with the lesion and surrounding vessel wall (ideally with minimal strain induced in the vessel and lesion), upon expansion and contact, an adhesive component will act as bioadhesive layer interfacing between the extracellular matrix of the native vessel and the device. This layer can be additionally released and activated resulting in structural linking of the components within the composite matrix both to one another and to the local tissue. The reconfigured matrix is relieved of tensile stresses induced during expansion, resulting in negligible or a slightly negative (compressive) load offering moderate radial support. Adhesion of the matrix to the thin fibrous caps common in vulnerable plaques and the surrounding tissue will provide local structural stiffening and support to prevent cracking and release of the necrotic lipid core. Optional biologically active components can be included within the base matrix of the prosthesis to further improve biological and vascular compatibility, promote healing and recruitment. The following embodiments demonstrate the scope and intent of the invention:

### EXAMPLE 1

In one embodiment, the matrix can include a structural material, a bioadhesive component and a bioactive component. The structural material is composed of a metallic alloy or a durable or bioabsorbable polymer that has very thin strut thickness and width is highly flexible and conforms to the vessel wall. The bioadhesive component is composed of one or several natural proteins resembling extracellular matrix proteins, mainly collagen or collagen derivates. This component is preferentially located on the outer abluminal surface of the device. The bioactive component is achieved through direct modification of the interior adluminal surface of the prosthesis. In a preferred embodiment, the adluminal surface modification is the application of an etched surface topography tailored for improved endothelial cell migration, growth, adhesion and maturation. In an alternate embodiment, the adluminal layer is a deposited surface coating for achieving the same purpose. Other combinations of surface application are possible and within the scope of the present invention.

### EXAMPLE 2

In another embodiment, the bioadhesive layer can act as the structural layer of the device. In this embodiment, the mixture of proteins must provide the structural support for the device. Blends of proteins such as collagen and elastin can be coupled with other compounds. These proteins can be assembled together to form tubes or preformed sheets that can be apposed to the vessel in-situ by an expandable delivery system such as a low-pressure balloon catheter. The bioadhesive component is deposited onto the external abluminal surface of the device to allow anchoring and apposition of the prosthesis to the vessel wall. This component will be preferentially located in the outer surface of the device but could be located throughout the entire surface of it

In a further derivation from the embodiments described above, the bioadhesive component incorporates molecules to allow bioactivation via secondary mechanism. These molecules could be incorporated via nanoliposomes, nanoparticles or any other carriers.

### DETAILED DESCRIPTION

The present invention seeks to fulfill the following desirable attributes by applying novel material composites, geometry and fabrication techniques to create a better prosthesis: (a) structural reinforcement of the thin fibrous cap; (b) mechanical compression, remodeling and therefore stabilization of the necrotic lipidic core; (c) radial reinforcement of the vessel structure across the entire circumference; (d) vascular conformability and flexibility to limit applied stresses and vascular injury from straightening and expansion both during and after deployment; and (e) promotion of vascular healing through modulation of inflammation, control of smooth muscle cell proliferation and promotion of endothelial cell growth.

Structural Layer. A structural layer is constructed from a mixture of biocompatible or biological materials that can be easily tolerated and readily reincorporated into the existing tissues of the vessel. Particular combinations will be limited by available techniques to synthesis and combine these materials in a manner which yields the demanding mechanical properties: as much as 500% radial expansion for delivery, resulting in a flexible compressive-load bearing structure once cross-linked at its expanded diameter. Stretchable biomaterials such as elastin could play a crucial role, possible in conjunction with more rigid load bearing scaffolds constructed of collagen or silk. The specific geometry of the biomaterial composite will play a crucial role on the eventual mechanical behavior at both the molecular level and at the scale of more visible features, similar to the complex strut geometry seen in stents.

The coronary arteries withstand and endure some cyclic strains from the pulsatile blood flow and motion of the beating heart. Once deployed, the thin structural matrix should provide only a negligible stiffening of the native vessel. Independent of the mechanism of expansion, at deployment the prosthesis will be tailored to expand the native coronary artery by no more than 25% at the most normal site and compress the plaque below the threshold of plaque rupture. By using this mechanism the prosthesis will cover, mold and remodel but tend not rupture the elastic components of the vascular wall. These properties can be controlled by providing a suitable combination of radial force and apposition which is depending upon the varying strut geometry and material utilized. Many variations in stent patterns and materials have been demonstrated in the prior art which can be tailored to achieve varying degrees of radial force.

Bioadhesive Component: An adhesive component is preferable for plaque passivation in accordance with the principles of the invention. It is important to emphasize that the bioadhesive component will bond the media of the vessel with the device's abluminal surface. By the nature of the material, the abluminal layer will enhance the incorporation of the device into the vessel wall. Adhesion between biomolecule components on the abluminal surface of the device and the vessel wall (including vessel wall proper and plaque within the vessel) may not occur immediately, but is expected to happen within 72 hours after device deployment, with full incorporation by 2 weeks. Preferably, adhesion is achieved via spontaneous or induced crosslinking or other joining or bonding of proteins between the prosthetic materials and native tissue. Therefore, the preferred embodiment is not based on the release of adhesive substances, but such release may be employed and is within the scope of the invention. In embodiments in which an adhesive is released, such release may for example be activated through the utilization of high strains seen during expansion, through the application of light based, ultrasonic or thermal energy or result from a chemical catalyst. In one embodiment, a thin layer or small packets (micro or nanospheres) of adhesive can be encapsulated and sealed within a stable material layer that is breeched during high strains of expansion. Once this layer is breeched, the adhesive is able to flow within the structural layers of the matrix and into the vessel wall.

In a preferred embodiment, the adhesive component is applied as either: (1) a thin coating, (2) sandwiched layers or (3) quilted layers—securing the encapsulant layer (top and bottom) in an array of small pockets across the surface. One alternate embodiment for fabrication of the quilted layers involves laser drilling a grid of holes through sandwiched coating layers to create small adhesive “spot” welds or stitches. Other options include stitching this layer to the structural layer with absorbable suture or biosilk. The bioadhesive layer will allow full incorporation of the structural component into the vessel wall by merging together the extracellular matrix components of the device and vessel wall. Also, this layer will provide additional fibrous cap reinforcement and the possibility for drug elution from the same matrix.

Bioactive Component. In order to further reduce the inflammatory response and promote quick natural healing with minimal neointimal growth, a bioactive component shall be provided as previously discussed. This biological process is achieved by either directly modifying the inner surface of the device or by adding nanoscale biological coatings to the surface. In a preferred embodiment, the inner surface of the device is modified to promote endothelial cell adhesion and colonization. The surface may include a nano-scale texture (e.g. wells, pits, raised bumps, protuberances, etc.) that promotes endothelialization, such as EC migration, adhesion and/or maturation, using for example, shallow surface feature depths on the order of 5 to 200 nanometers and lateral feature aspects on the order of 50 nm to 5 microns and a coverage of approximately 1 to 500 features per 10 μm².

In a further embodiment, the entire matrix is coated in albumin in order to reduce the immune response. In another embodiment, a coating may include proteins that selectively deter undesirable proteins and selectively promote the adhesion and incorporation of desirable endothelial cells on the surface of the device. For example, the present invention may employ the techniques of U.S. Pat. No. 7,037,332 and/or U.S. Publication No. 2004/0170685, which disclose coating with proteins that attract/bind to endothelial cells and/or endothelial cell precursors (EPC) to promote endothelialization of an implant and which are each incorporated by reference herein. The physical surface features promoting endothelialization and the biomolecule coating promoting endothelialization may be combined on the same surface. As defined herein, the terms “endothelialization-promoting” and “endothelial cell-promoting” include one or more of: recruiting endothelial cells or their precursors by binding said cells or promoting the growth, proliferation, survival, attachment and/or residence of said cells. Therapeutic drug eluting layers may also be provided to further control the healing process. Drug release may result from degradation of a natural polymer layer, diffusion from porous surfaces, etc. Suitable drugs include but are not limited anti-proliferative agents such as conventional stent based antiproliferative agents.

Coatings of the invention may be formed by any suitable method, such as those known in the art. For example, the coating methods disclosed in U.S. Pat. Nos. 5,516,703; 5,728,588; 5,851,230; 6,153,252; 6,284,503; 6,670,199; 6,087,452; 6,913,617 and U.S. Pub. No. 2005/244456, each of which is incorporated by reference herein, may be used for coating surfaces according to the present invention.

Matrix Properties. The prostheses must contain certain mechanical, biological and technical features in order to accomplish the goal of sealing and passivating atherosclerotic plaques at risk of disruption.

From the mechanical point of view, the matrix should retain low to intermediate circumferential radial force after expansion. In its final constructed shape, the total barrier thickness may range from 0.0020″ to 0.1.″ The forces applied by the matrix will be enough to keep the vessel open but not significant to cause continuous vessel stress. The vascular prosthesis may, for example, impose expansion forces in the range of 30 to 250 mm Hg—and these forces can be modified according to the type of plaque that will be treated. If self-expandable, the vascular prosthesis should have higher radial forces at the borders, where shoulder stabilization is required. Also, by function of the structure, these mechanical properties may allow better positioning and anchoring of the prosthesis to the vessel wall. After expansion and anchoring of the matrix, the final three-dimensional structure preferably does not significantly deviate from the natural angulation of the vessel. In one variation, the deviation is no more than 10 degrees.

Several factors will impact on the biological properties of the matrix. Primarily, the matrix will be constructed out of biocompatible and bioabsorbable natural components combined in the various ways described. The final composite should retain anti-thrombotic properties. An alternate embodiment of the invention utilizes materials which have the capability of absorbing one or several medications rendering the matrix with anti-inflammatory and anti-proliferative properties. Once constructed, the milieu will serve as a culture media for cell capturing, seeding and nesting promoting healing of the intervened vascular segment.

The invention offers significant technical advantages compared to current available technology. The matrix may maintain a very low unfolded or collapsed profile of less than 800 nanometers. In other embodiments, a broader range of sizes for arterial (medium), peripheral (large) and neural (small) vessels may be useful, perhaps in the range of 0.5 mm to 10 mm. This prosthesis could also be suitable in size as large as 60 mm for treating other endovascular diseases such as aortic aneurisms, or thoracic diseases and disorders.

Vascular prosthesis components. Although variation of the matrix may occur, the basic principle is the one of building a milieu similar to the ECM which will enable the vessel to recruit cells and promote healing following matrix deployment. Therefore, this matrix can be viewed as a milieu or culture media for cells to attach and grow. However, some radial force is needed in order to maintain the vessel patent after the matrix is deployed into the vessel wall. The luminal prosthesis is constructed from an array of materials in accordance with the principles of the invention. These materials can include: implant grade metals, durable polymers, erodible and bioabsorbable polymers, biomolecules and pharmaceutical compounds.

Balloon expandable stents expand and then retain their radial strength via the ductility of stainless steel or other biocompatible structural material. A possible embodiment includes a thin strut metallic scaffold to be used as part of the composite to achieve radial strength for minor dilation. The anchored device should be designed to yield minor radial strength compared to a metallic stent. Biomaterials and biodegradable polymers are much more flexible than steel, and the radial strength suffers as a result, by several orders of magnitude.

A suitable biomaterial matrix may be formed by reconstructing, at least in part, what is found in existing structures in nature. For blood vessels, one place to look is the extracellular matrix of the basal lamina reticulum. The basal lamina reticulum consists of segments of Type IV collagen associated through various available bonding sites (N-terminal, C-terminal and lateral association) bound with the multi-adhesive matrix protein laminin, entactin, fibronectin and various proteoglycans, including hyaluranon (hyaluronic acid) and heparin sulfate. Additional Types III and/or VI fibrous collagen can be included to offer further structural support. These constituents can be mixed in varying ratios to yield the desired properties. These materials can also be combined in various ways with the other materials mentioned above to yield the desired biological and mechanical properties. As outlined above, the basic principle is the one of building a milieu similar to the ECM that will enable the vessel to recruit cells and promote healing following matrix deployment. Therefore, this matrix can be seen as a milieu or culture media for cells to attach and grow.

The materials used to construct this prosthesis will vary according to the specific function or characteristics of any specific structural component. The basic skeleton of the will require a material that supports the continuous mechanical compression of the vessel. This backbone will tolerate the bending and torsional forces imposed by heart beating. Implant grade metallic components such as 316L stainless steel and Nitinol have been shown to provide adequate radial forces, scaffolding and mechanical support in the form of balloon expandable stents, with strut widths ranging from 50 to 500 microns. Mechanical properties of these materials are also highly dependent upon the strut geometry. Durable polymers (Polycarbonate, ABS, Nylon, Polyester, etc.) have not proved to be as functional when used as the structural material in a stent, primarily due to the larger strut thicknesses required to supply adequate support which further worsens the biological and vascular compatibility of these materials for implantation. These materials have been widely utilized for drug release, a property which the present invention would also benefit from.

TABLE 1
Yield Strength, Young's Modulus and Elongation
at Yield for various engineering materials.

| Material Name | Elastic Modulus (E) | | Yield Strength (S_{y}) | | Elongation |
|---|---|---|---|---|---|
| 316L Stainless Steel | 195 | GPa | 500-1500 | Mpa | .2-.4% @ yield |
| | | | | | 4-57% @ break |
| Nitinol | (austenitic) 75 GPa | | (austenitic) 560 Mpa | | 5-17% @ break |
| | (martensitic) 28 Gpa | | (martensitic) 100 Mpa | | <8% @ yield |
| ABS | 1.8-3.2 | GPa | 30-65 | MPa | 1.7-6% @ yield |
| | | | | | 2-110% @ break |
| Polycarbonate | 1.6-2.4 | GPa | 58-70 | MPa | 6-8% @ yield |
| | | | | | 8-135% @ break |
| Polyurethane | <2 | GPa | <35 | MPa | 8-11% @ yield |
| | | | | | 10-850% @ break |
| PLGA | 3.3-7.0 | GPa | <30 | MPa | 4% @ yield |
| | | | | | 6% @ break |
| Collagen | 1-200 | MPa | — | | 5-10% |
| Synthesized | 0.2-1.03 | MPa | 30-800 | kPa | 140-150% |
| Collagen(I)-Elastin- | | | | | |
| Chondroitan Sulfate | | | | | |
| Tissue | | | | | |

A comparison of the mechanical properties of the available materials for prosthesis design is useful for determining the proper choice and proportions of materials for a useful luminal prosthesis. The strongest yet least flexible materials available are ceramics. Ceramics achieve high elastic moduli.

A suitable metallic component includes but is not limited to one of the following: stainless steel alloys, 316L stainless steel, Nickel-Titanium alloys (Nitinol), Titanium, Titanium alloys, cobalt-chromium alloys, tantalum, niobium, and niobium alloys.

Suitable durable polymeric components include but are not limited to one or more of the following: polyurethane, PVP, polyethylene, Acrylic, PBMA, PEVAMA, polyester, hydrogels, polyimide, polyamide, parylene and parylene derivatives.

Suitable erodible and bioabsorbable polymers include but are not limited to one or more of the following: catgut, siliconized catgut, chromic catgut, Polyglycolic Acid (PGA), Polylactic Acid (PLA), copolymers of PLA/PGA, Polydioxanone, Polycaprolactone, Polyhydroxybutyrate (PHB), polyethylene terephthalate (PET/Polyester), polyethylene terephthalate-glycolide copolymer, photopolymerized polyvinyl alcohol gels.

Bioadhesive layer or bonding layer coating: Elastic and resistant layer either coating the skeleton or conforming parallel fibers covering the skeleton will be incorporated to provide mechanical support and allow bonding to the components to the media of the vessel. Structural proteins, including collagen, chitosan and elastin and specialized proteins, including fibrillin, Tenascin, Entactin, Thrombospondin, integrin, litegrin can be used.

Proteoglycans and Glycosaminoglycans (GAGs), including heparin and heparin sulfates (perlecan, syndecan), hyaluron and hyularonates, dermatan sulfates, chondroitan sulfate, keratan sulfates; lipid-based compounds including, myristic acid, palmitic acid, palmitoleic acid, stearic acid, oleic acid, linoleic acid, linolenic acid and arachidonic acid; biopolymers, including alginate, cellulose, spider silk; multi-adhesive matrix proteins: laminin, fibronectin, cadherins, N-Cams. In this particular component of the matrix, compounds can be incorporated for drug elution. Pharmaceutical compounds may be optional, but are likely to help promote healing or otherwise alter the vascular response to prevent restenosis, thrombus formation or other unwanted effects. Suitable pharmaceuticals include (but are not limited to) paclitaxel, heparin, sirolumus and tacrolimus and other—limus derivatives, mitomycin C, antibiotics or other anti-proliferatives or anti-inflammatory agents.

Bioactive layer: An inner coating anchoring layer/coating can be used to avoid the non-selective adhesion of serum proteins and promote the adhesion of endothelial cell precursors of mature endothelium. In its simplest form, the surface of the structural matrix can be modified by nanoscale texturing (abrasive etching, chemical etching, electrochemical etching, electropolishing, ion-beam, plasma or other CVD/PVD derived etching and deposition processed, electroplating, and de-alloying. One or more of these processes may be required in various combinations to generate the desired surface topography and biocompatible chemistry.

For example, the surface of a Nitinol self-expanding coronary stent may be modified using an etching process to create a stippled surface resembling orange peel with surface features approximately 300 to 1000 nm across and 20-50 nm in height spaced evenly over the entire surface with a relative uniform surface density of approximately 50%. The stippled texture is smooth and undulating, with no sharp edges. A nano-textured surface of the same Nitinol surface may be obtained by sandblasting with small (1 μm or lower) grit media, then electropolishing to an average peak-to-peak roughness of 20-50 nm. Generally, methods for obtaining surface texture include but are not limited to magnetron sputtering, chemical etching, electro-chemical etching, abrasive tumbling, abrasive media blasting, sanding, scratching, laser etching, atomic layer deposition (ALD), chemical vapor deposition (CVD) and physical vapor deposition (PVD) technologies alone or in combination and other technologies commonly employed for the fabrication of MEMS devices and computer chip fabrication technology. Use of a mask for controlling feature size, shape and distribution is also possible during the processes described above. Masking options include but are not limited to spray-on resists, photo-cured resists and other technologies commonly employed for the fabrication of MEMS devices and computer chip fabrication technology. The textured surface can be applied directly to the base substrate material or as an applied metallic, ceramic, polymeric, or biological coating. U.S. Publication Nos. 2006/0004466 and 2006/0121080 each disclose surface modification methods that may be used and are each incorporated by reference herein. The surface features may for example be depressions, such as wells or pits, or may be raised features, such as, islands or “bumps.”

An additional outer bonding agent layer/coating may be used to cross-link the deployed vascular prosthesis. Compounds such as crosslinker—pyridinoline, 1-ethyl-3-(3 dimethyl aminopropyl) carbodiimide (EDC), N-hydroxysuccinimide (NHS), nafthalimide can be included and activated by light, laser energy, temperature changes, pressure changes or other means.

These materials can be combined in many different ways to form a structure suitable for vascular implantation. A dissolved slurry of one or more components above (excluding the metals and durable polymers) can be created and deposited, extruded or molded into an appropriate shape. Suitable shapes include tubes and flat films which can be rolled into tubes. More complex geometry may be possible through specialized processing (CNC laser cutting, deposition, spinning or weaving) or tooling (patterned molds) to enhance physical properties. Multiple layers can also be combined, interwoven, stacked or directly deposited onto one another with each layer yielding varying properties suitable to its function relative to the other layers and location in the anatomy. The geometry of each layer can vary as well to tailor each materials function to its role in the overall matrix. Various geometrical patterns such as those found in stents to provide the desired amount of radial force, flexibility, expandability, structural coverage, and drug elution coverage.

Further refinements in these scaffolds is possible with the application of computer-numerical-controlled (CNC) three dimensional deposition, also referred to as 3D inkjet printing. The physical properties of a raw elastin-collagen scaffold could be further enhanced by computer-directed deposition of the cross-linking compounds. For example, NHS or EDC printed as an array of lines onto the raw scaffold can impart enhanced elasticity in a specified direction. This property can be exploited to create an expandable stent-like scaffold which can be delivered to a desired site in a small profile delivery system catheter and then expanded and anchored at the site.

Matrix Fabrication. A discussion of fabrication methods can be broken down into two sections. First, there is fabrication of individual component structures. Secondly, there is the assembly of these scaffolds into a single composite scaffold or matrix. In general, it is desirable to construct the final composite into a tubular form. Certain techniques are well suited to the manufacture of tubular structures. Other approaches may be better suited to working with flat planar geometry with a subsequent rolling process to create a tube form.

Fabrication methods for the component structures can be tailored to fit the needs of specific materials chosen. For example, a structural metallic component may be formed by laser cutting of a polished tube. Another possibility for metallic components is wire forms, bent, fused and cut into desired patterns. Both of these techniques have been used for marketed stents and stent grafts. A still further possibility is the controlled deposition of metal through sputtering or extrusion. Deposition and coating processes may be utilized for making thin films and coating.

Three-dimensional printing, such as the process available from Microfab, Inc. ‘(Plano, Tex.) has matured considerably in the last decade. The basic premise is that a computer based CAD model can be processed in such a way to instruct the motion of a printing head in three dimensions relative to a base substrate. Inkjet printers have become a commodity market and can deposit complex two dimensional patterns with various inks with high resolutions enabling feature sizes on the scale of tens of microns. Addition of a third dimension to the relative mobility of the print head is used in rapid prototyping equipment, where actual inkjet printer-based printer heads deposit adhesives and inks to powder resins which are stacked one layer at a time to build complex forms in an array of impressive colors. An example of a 3-D printer is Z-Corp's Z406 Printer.

Matrix Anchoring. Because of the atraumatic nature of the device, circumferential support force should be considered. Therefore, a mechanism which allows permanent and complete apposition of the prosthesis onto the plaque surface needs to be incorporated. Traditional metal stenting creates scaffolding with relatively high radial forces. Such radial forces are ultimately undesirable as they lead to increased injury to the vessel wall as evidenced by restenosis. Such radial forces are eliminated when lower forces are required to expand the prosthesis, although another method of fixation is required to prevent migration and collapse of the stent. If a self-expandable structure is used, the vascular prosthesis must posses a structural mechanism that allows the edges of the device to anchor at the borders of the plaque therefore stabilizing the shoulders where the strain forces are the highest and slightly compressing the center of the lesion where the plaque components are more abundant (Figure). While other more traumatic options are available such as stapling, suturing, crimping, etc., a less invasive and non-toxic adhesive type bond is preferred. The novel prosthesis therefore incorporates an adhesive layer affixed to, deposited onto or incorporated within the structural matrix. Examples of potential adhesives include: Gelatin, redu-formalin (GRF), photosensitive glues, vitamin E, cyanoacrylate, photosensitive acrylics, nafthalimide (crosslink with vessel tissue).

Fixation to the vessel may be provided by one or more of the following mechanisms: (1) curing, binding, cross linking of molecular bonds, proteins, etc. via applied light energy (example: 405 nm light and Naftalimide); (2) curing, binding, cross linking of molecular bonds, proteins, etc. via thermal energy (applied, removed or locally available); (3) curing activated from contact with local tissues and fluids (e.g., water); (4) adhesive, catalyst or activator delivered locally via permeable balloon; (5) locally delivered adhesive agent (cyanoacrylate, UV cured acrylic,) via permeable balloon; (6) strain induced curing or work hardening from expansion; (7) Regrowth through biological process; and (8) incorporation of the matrix and native proteins and cholesterol.

Preferred Embodiments: Device/Utility: Component mixes: (1) Collagen IV, Elastin, Hyaluran Acid (HA)+basic cross-linker (NHS/EDC); (2) Collagen IV, Collagen III, Elastin, HA+basic cross-linker (NHS/EDC); (3) Coll IV, Elastin, HA and Naftalimide or other in-situ light activated cross linker; (4) Collagen IV, Elastin, HA & PLGA and (5) Structure geometry.

Method I—Fabrication options include but are not limited to: (1.) Flat film sandwich rolled onto delivery balloon; (2) Self expanding tube; (3) single roll; (4) multi-roll (5) electrospinning and (6) flat film molding.

The following disclosures are incorporated herein by reference in their entireties: U.S. Pat. Nos. 6,176,871; 6,087,552; 6,667,051; 6,632,450; 6,372,228; 6,110,212; 6,087,552; 5,990,379; 5,989,244; 6,004,261; 5,100,429; 6,669,721; 6,666,882; 4,575,330; 5,334,201; 5,410,016; 6,626,863; 5,334,201; 5,410,016; 5,626,863; 5,609,629; 5,443,495 Esen et al, “Preparation of monodisperse polymer particles by photopolymerization”, J Colloid Interface Sci 179:276-280 (1996) (Abstract only); Hayashi et al, Elastic properties and strength of a novel small-diameter, compliant polyurethane vascular graft”, J. Biomed. Mater. Res.: Applied Biomaterials, 23(A2):229-224 (1989); Hill-West et al, “Inhibition of thrombosis and intimal thickening by in situ photopolymerization of thin hydrogel barriers”, Proc Natl Acad Sci USA 91:5967-5971 (1994); and “Polymeric Endoluminal Paving”, Slepian (Cardiology Clinics 12(4):715-737, 1994).

One objective of the bioadhesive abluminal layer is to provide a microenvironment similar to the one provided by the extra-cellular matrix components of the vascular wall. This bioadhesive component may be composed of one or several combinations of proteins including collagen, elastin, fibronectin, laminin, glycosaminoglycans (GAGs) and proteoglycans. There are a variety of components and combinations thereof that may be included according to the invention. Accordingly the examples provided herein are for the purposes of illustration and do not limit the invention.

### EXAMPLE 3

The structural layer or skeleton may be an ultra-thin self-expandable Nitinol alloy with a specific configuration in which the skeleton is covered by a thin bioadhesive component. In a preferred embodiment, this bioadhesive component includes or is composed of collagen. The layer may have a thickness of from 400 nm to 120 microns (enough to reinforce the thickness of the thinned fibrous cap). The average fiber size may be 100 to 800 nm and the average porosity size is preferably from 1 to 20 microns, enough to allow cell seeding and protein incorporation. The collagen layer may have a degradation time of less than 2 weeks, reaching 50% degradation in less than 4 days. In this embodiment, the coating may be disposed around the struts (all surfaces) or can cover only the abluminal side of the prosthesis. In a preferred variation, the inner surface of the device is modified to allow endothelial cell adhesion and colonization. This biological process is achieved by either directly physically modifying the inner surface of the device and/or by adding Nanoscale biological coatings to the surface. Thus, the surface may have or include a nano-scale texture (e.g. wells, pits, raised bumps, protuberances, etc.) that promote EC migration, adhesion and maturation, preferably with shallow surface feature depths on the order of 5 to 200 nm and lateral feature aspects on the order of 50 nm to 5 microns and a coverage of approximately 1 to 500 features per 10 μm². Nanocoatings in the range of 1 to 500 nm in thickness of proteins such as fibronectin, vitronectin, albumin, RGD peptides, modified polymers or specific antibodies may also be applied on top of the Nanotexture to enhance cell recruitment by the prosthesis.

### EXAMPLE 4

The structural layer or skeleton may be an ultra-thin self-expandable

Nitinol alloy with a specific configuration in which the skeleton is covered by a thin bioadhesive component. In a preferred embodiment, this bioadhesive component includes or is composed of elastin. The average fiber size may be 100 to 800 nm and the layer may have a thickness of from 400 nm to 120 microns (enough to reinforce the thickness of the thinned fibrous cap) and an average porosity of 10 to 120 μm. The elastin coating may have a degradation time of less than 2 weeks, reaching 50% degradation in less than 4 days. In this embodiment, the coating may be disposed around the struts (all surfaces) or can cover only the abluminal side of the prosthesis. In a preferred embodiment, the inner surface of the device is modified to promote endothelial cell adhesion and colonization. This biological process is achieved by either directly physically modifying the inner surface of the device and/or by adding Nanoscale biological coatings to the surface. Thus, the surface may have or include a nano-scale texture (e.g. wells, pits, raised bumps, protuberances, etc.) that promotes EC migration, adhesion and/or maturation, preferably with shallow surface feature depths on the order of 5 to 200 nanometers and lateral feature aspects on the order of 50 nm to 5 microns and a coverage of approximately 1 to 500 features per 10 μm². Nanocoatings in the range of 1 to 500 nm in thickness of proteins such as fibronectin, vitronectin, albumin, RGD peptides, modified polymers or specific antibodies may also be applied on top of the Nanotexture to enhance cell recruitment of the prosthesis.

### EXAMPLE 5

The structural layer or skeleton may be an ultra-thin self-expandable

Nitinol alloy with a specific configuration in which the skeleton is covered by a thin bioadhesive component. In a preferred embodiment, this bioadhesive component includes or is composed of a mixture of elastin and collagen. The proportions may be adjusted according to the objective of the matrix to be constructed. For example, 80-90% collagen and 10-20% elastin may be used if vascular support is required and 50-70% collagen and 30-50% elastin may be used if more elasticity is desired. It is conceived that one or several polymers or other biological materials may also be included in order to make the mixture more stable. In any case, elastin and collagen should be mixed but ideally the collagenous material should preferentially be located in the abluminal aspect of the device. The average fiber size may be 100 to 800 nm and the layer may have a thickness of from 400 nm to 120 microns (enough to reinforce the thickness of the thinned fibrous cap). The composite coating may have a degradation time of less than 2 weeks, reaching 50% degradation in less than 4 days. In a preferred embodiment, the inner surface of the device is modified to allow endothelial cell adhesion and colonization. The device may be coated in any of the manners described herein and may also be provided with nano-scale textural features in any of the manners described herein.

### EXAMPLE 6

The entire structural layer or skeleton may be composed or an ultra-thin self-expandable or balloon-expandable bioadhesive layer. In a preferred embodiment, this bioadhesive component includes or is composed of a mixture of elastin and collagen. The proportions may be adjusted according to the objective of the matrix to be constructed. For example, 80-90% collagen to 10-20% elastin may be used if vascular support is required and 50-70% collagen and 30-50% elastin may be used if more elasticity is sought. It is conceived that one or several polymers or other biological materials can be included in order to make the mixture more stable. The following combinations may, for example, be used: (1) Collagen IV, Elastin, Hyaluran Acid (HA)+basic cross-linker (NHS/EDC); (2) Collagen IV, Collagen III, Elastin, HA+basic cross-linker (NHS/EDC); (3) Coll IV, Elastin, HA and Naftalimide or other in-situ light activated cross linker; (4) Collagen IV, Elastin, HA & PLGA. In any case, elastin and collagen should be mixed but ideally the collagenous material should be preferentially located in the abluminal aspect of the device. The layer should have a thickness from 400 nm to 120 microns (enough to reinforce the thickness of the thinned fibrous cap). The composite may have a degradation time of less than 12 weeks. Cross-linking of the coating components may be necessary to achieve the desired radial forces. In a preferred embodiment, the inner surface of the device is modified to promote endothelial cell adhesion and colonization. The device may be coated in any of the manners described herein and may also be provided with nano-scale textural features in any of the manners described herein.

Various aspects of the invention are further described below with reference to the appended figures.

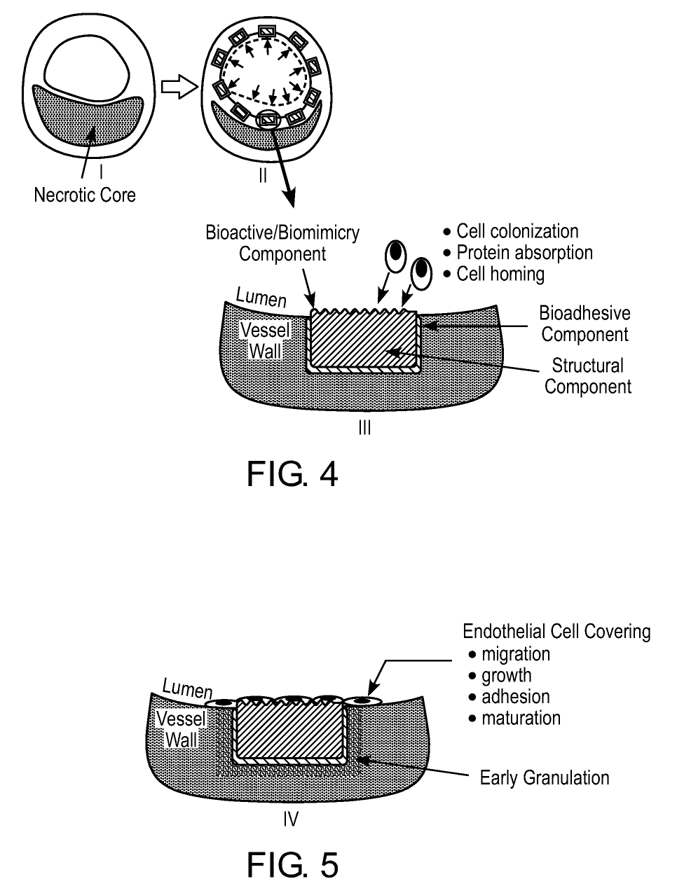

FIG. 4 illustrates an initial mechanical stabilization phase in the response of a blood vessel to treatment with a prosthesis embodiment of the invention. The prosthesis has been expanded at the site of treatment in the blood vessel and the struts of the prosthesis have begun to protrude into the vessel wall. The adluminal face of the prosthesis has not yet been colonized by endothelial cells.

FIG. 5 illustrates a further phase in the response of a blood vessel to treatment with a prosthesis embodiment of the invention. The struts of the prosthesis have protrude further into the vessel wall and the adluminal surface of the prosthesis has been colonized by endothelial cells. Early granulation is also seen in the vessel surround the bioadhesive component surface(s) of the prosthesis.

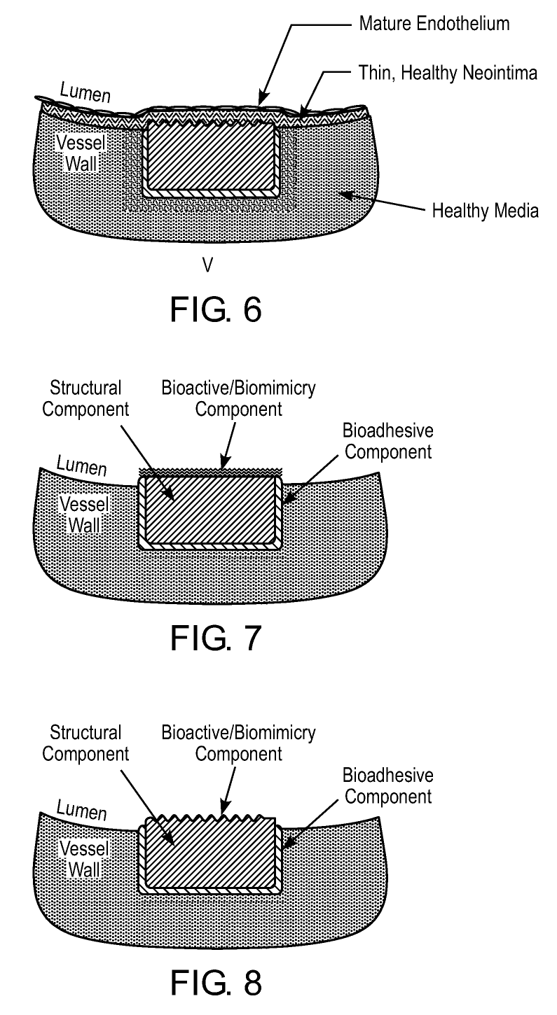

FIG. 6 illustrates a next phase in the response of a blood vessel to treatment with a prosthesis embodiment of the invention. Here a new thin, healthy neointimal surface has formed overlaid by a mature endothelial layer that has been established.

FIG. 7 illustrates an embodiment of a composite vascular prosthesis according to the invention. The embodiment includes a structural component coated on the adluminal face with a bioactive component and coated on its abluminal and side faces with a bioadhesive component.

FIG. 8 illustrates an embodiment of a composite vascular prosthesis according to the invention. The embodiment includes a structural component having endothelialization-promoting adluminal surface structural features and coated on its abluminal and side faces with a bioadhesive component.

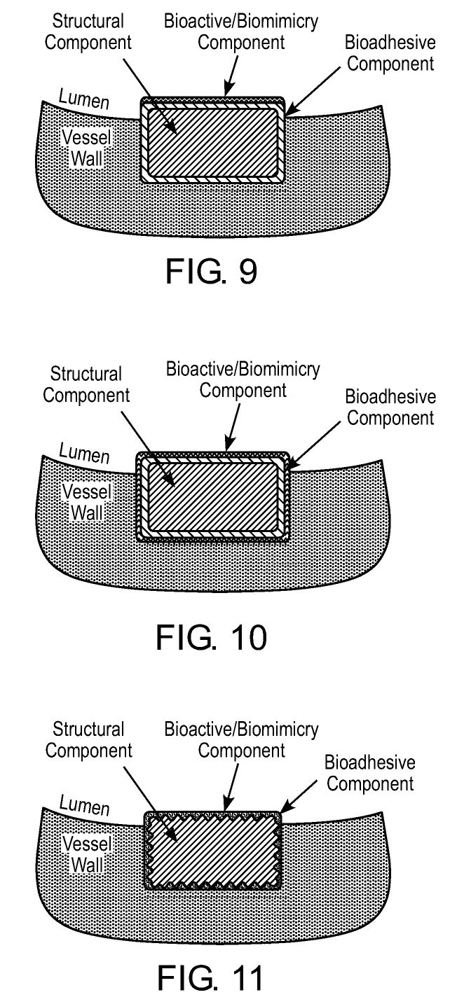

FIG. 9 illustrates an embodiment of a composite vascular prosthesis according to the invention. The embodiment includes a structural component coated on all its surfaces (sides) with a bioadhesive component and further coated on its adluminal surface with an endothelialization-promoting bioactive coating.

FIG. 10 illustrates an embodiment of a composite vascular prosthesis according to the invention. The embodiment includes a structural component coated on all its surfaces with a bioadhesive component which is then further coated on all surfaces with an endothelialization-promoting bioactive coating.

FIG. 11 illustrates an embodiment of a composite vascular prosthesis according to the invention. The embodiment includes a structural component having endothelialization-promoting surface features on all of its sides and which is also coated on all of its sides by a bioadhesive component.

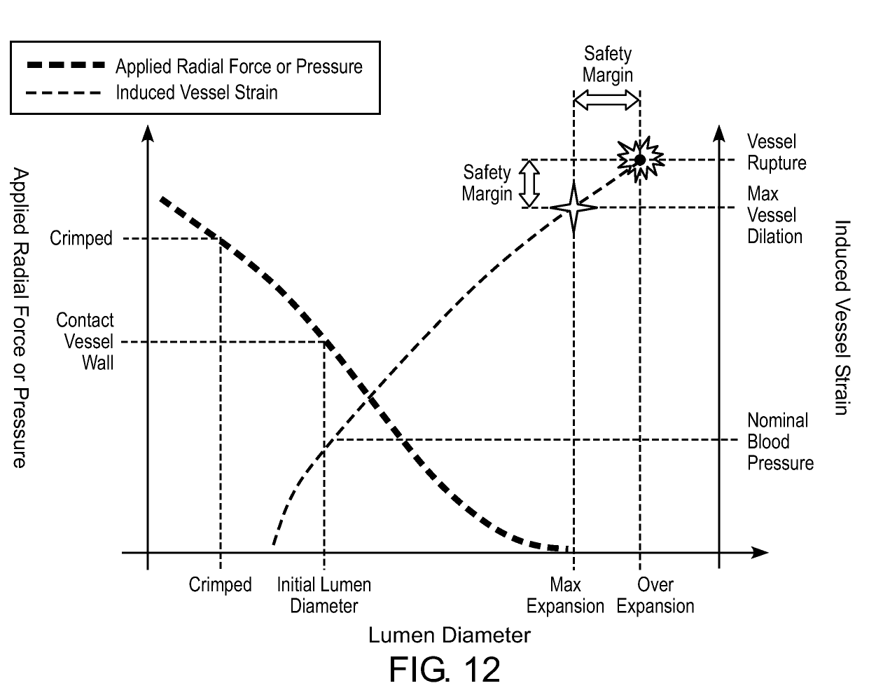

FIG. 12 is a graph illustrating the relationship between induced vessel strain, applied vessel force or pressure and lumen diameter. A safety zone is identified for treatment.

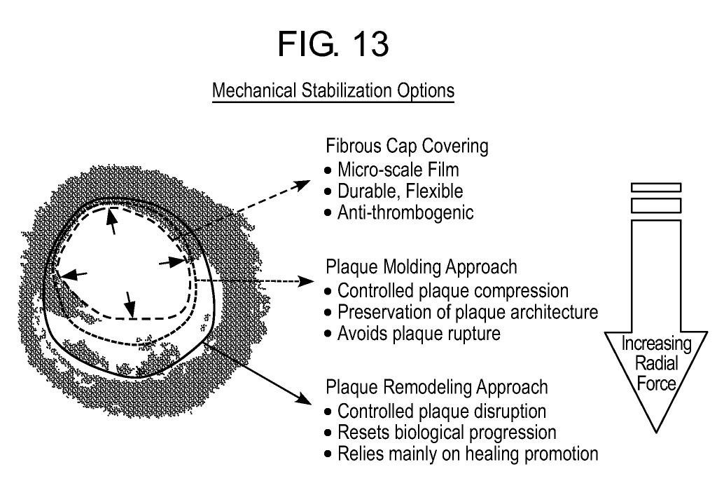

FIG. 13 illustrates various mechanical stabilization approaches that vary in the extent to which radial force is applied to an atherosclerotic lesion. At the low end of radial force is, for example, treatment of vulnerable plaque characterized by a fibrous cap. In this approach, for example, a micron-scale film that is durable and flexible and antithrombotic may be used for treatment. In a mid-range of radial force is a plaque molding approach characterized by controlled plaque compression, preservation of plaque architecture and avoiding plaque rupture. At the high end of radial force is a plaque remodeling approach characterized by plaque disruption and re-setting of biological progression of plaque, which relies mainly on promoting a healing response.

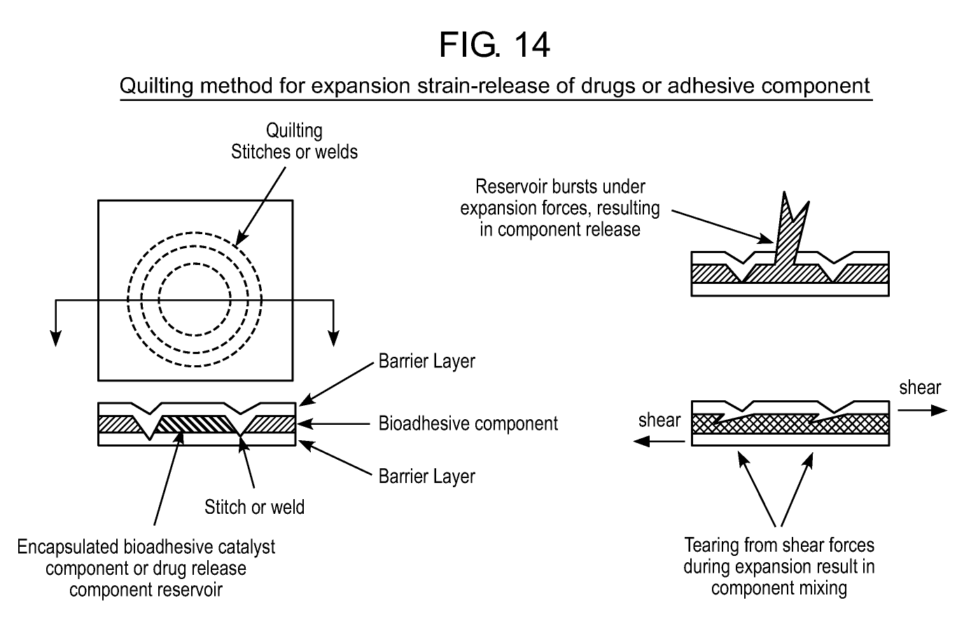

FIG. 14 illustrates a quilting method embodiment for expansion strain-release of drugs or adhesives. Compartments capable of containing drugs and/or adhesives are formed in layers of a prosthesis by a “quilting” approach. Under the forces of expansion of the prosthesis, the compartments may burst resulting in release of their contents and/or neighboring compartments may open to each other resulting in the mixing of their contents. In one embodiment, the layer that bursts is disposed on the abluminal face of the prosthesis so that drugs and/or adhesive components will be directed to a blood vessel wall during deployment of the prosthesis.

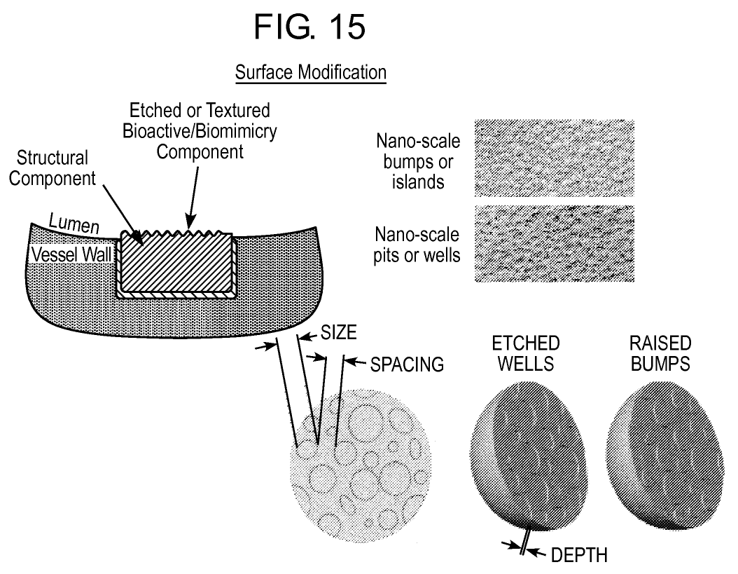

FIG. 15 illustrates various structural surface modification aspects of the prostheses of the invention. At least the adluminal face may be surface modified or, for example, only the adluminal face may be so modified as shown in the figure. As further shown, the surface structural features may take the form of depressions or raised features.

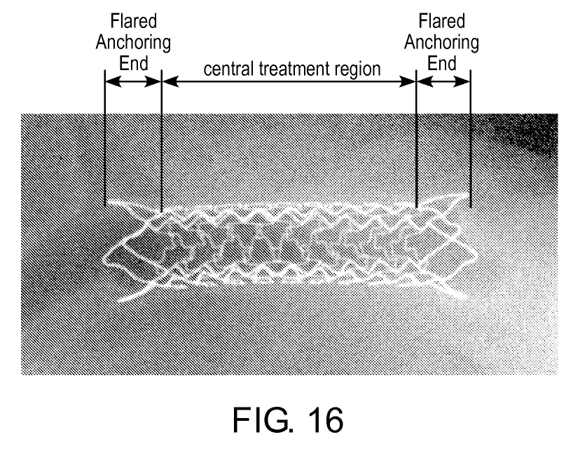

FIG. 16 illustrates a stent design that may serve as a structural component for a composite vascular prosthesis according to the invention. The stent design has a central treatment region and two flared ends. The flared ends inhibit lateral migration of a deployed prosthesis in a blood vessel.

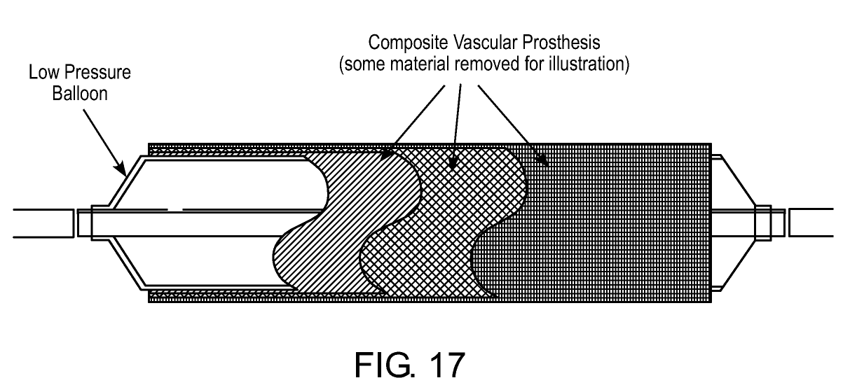

FIG. 17 illustrates a composite vascular prosthesis embodiment of the invention that consists of three layers, i.e., an adluminal bioactive layer, a structural layer and an abluminal adhesive layer, mounted on a low-pressure balloon delivery catheter.

Any of the treatment methods of the invention may include a step of locating an atherosclerotic lesion, such as a vulnerable plaque lesion, to be treated by the prosthesis in a patient. According to the invention, determining the location of a vulnerable plaque or other type of atherosclerotic lesion in a blood vessel of a patient can be performed by any method or combination of methods. For example, catheter-based systems and methods for diagnosing and locating atherosclerotic lesions can be used, such as those employing optical coherent tomography (“OCT”) imaging, temperature sensing for temperature differentials characteristic of vulnerable plaque versus healthy vasculature, labeling/marking vulnerable plaques with a marker substance that preferentially labels such plaques, infrared elastic scattering spectroscopy, and infrared Raman spectroscopy (IR inelastic scattering spectroscopy). U.S. Publication No. 2004/0267110 discloses a suitable OCT system and is hereby incorporated by reference herein in its entirety. Raman spectroscopy-based methods and systems are disclosed, for example, in: U.S. Pat. Nos. 5,293,872; 6,208,887; and 6,690,966; and in U.S. Publication No. 2004/0073120, each of which is hereby incorporated by reference herein in its entirety. Infrared elastic scattering based methods and systems for detecting vulnerable plaques are disclosed, for example, in U.S. Pat. No. 6,816,743 and U.S. Publication No. 2004/0111016, each of which is hereby incorporated by reference herein in its entirety. Temperature sensing based methods and systems for detecting vulnerable plaques are disclosed, for example, in: U.S. Pat. Nos. 6,450,971; 6,514,214; 6,575,623; 6,673,066; and 6,694,181; and in U.S. Publication No. 2002/0071474, each of which is hereby incorporated herein in its entirety. A method and system for detecting and localizing vulnerable plaques based on the detection of biomarkers is disclosed in U.S. Pat. No. 6,860,851, which is hereby incorporated by reference herein in its entirety. Angiography using a radiopaque and/or fluorescent dye, for example, as known in the art, may be performed before, during and/or after the step of determining the location of the vulnerable plaque, for example, to assist in positioning the prosthesis in a subject artery.

Each of the patents and other publications cited herein is incorporated by reference as if set forth in its entirety herein.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. An expandable vascular prosthesis, comprising:
an at least substantially tubular, radially expandable structural component comprising an abluminal surface and an adluminal surface; and
an adhesive coating comprising at least one molecule selected from the group consisting of a collagen and an elastin, wherein the adhesive coating is disposed on at least part of the abluminal surface of the structural component, and
wherein the adluminal surface comprises surface features having depths in the range of 5 nm to 5 μm and lateral dimensions in the range of 50 nm to 5 microns, said surface features being present on the adluminal surface at a density of 1 to 500 surface features per 10 μm².

2. The prosthesis of claim 1, wherein at least part of the adluminal surface is coated with at least one biomolecule.

3. The prosthesis of claim 2, wherein the at least one biomolecule coated on the adluminal surface comprises a fibronectin.

4. The prosthesis of claim 1, wherein the bioadhesive coating comprises an activatable protein crosslinker.

5. The prosthesis of claim 1, wherein the prosthesis is self-expanding.

6. The prosthesis of claim 1, wherein the structural component is metallic.

7. The prosthesis of claim 1, wherein the structural component is polymeric.

8. The prosthesis of claim 1, wherein the prosthesis exerts a radial expansion force in the range of 30 to 750 mm Hg in a radially expanded state.

9. The prosthesis of claim 8, wherein the prosthesis exerts a radial expansion force in the range of 30 to 250 mm Hg in a radially expanded state.

10. The prosthesis of claim 1, wherein the structural component has a wall thickness in the range of 20-100 microns.

11. The prosthesis of claim 1, wherein the adluminal surface comprises surface features having depths in the range of 5 nm to 200 nm

12. A method for treating an atherosclerotic lesion in a blood vessel of a patient, comprising the steps of:
locating a site of an atherosclerotic lesion in a blood vessel of a patient;
transporting a prosthesis according to claim 1 in an unexpanded state to the site of the atherosclerotic lesion in the blood vessel; and
radially expanding the prosthesis at the site of the atherosclerotic lesion so that the prosthesis contacts the blood vessel at the site.

13. The method of claim 12, wherein the atherosclerotic lesion is a vulnerable plaque.

14. The method of claim 12, wherein the atherosclerotic lesion is an atherosclerotic lesion freshly treated by angioplasty.

15. The method of claim 12, further comprising the step of:
crosslinking the bioadhesive coating of the prosthesis to the blood vessel.

16. The method of claim 15, wherein the bioadhesive coating of the prosthesis further comprises an activatable crosslinker and the step of crosslinking the bioadhesive layer of the prosthesis to the blood vessel comprises activating the activatable crosslinker.

17. A radially expandable vascular luminal prosthesis, comprising:
a structural component;
an adhesive abluminal surface; and
an endothelial cell-promoting adluminal surface.

18. The prosthesis of claim 17, wherein the prosthesis exerts a radial expansion force in the range of 30 to 750 mm Hg in a radially expanded state.

19. The prosthesis of claim 18, wherein the prosthesis exerts a radial expansion force in the range of 30 to 250 mm Hg in a radially expanded state.

20. The prosthesis of claim 17, wherein the adhesive abluminal surface is conditionally adhesive.

21. The prosthesis of claim 17, wherein the adhesive abluminal surface comprises at least one protein providing adhesiveness of the prosthesis to a blood vessel wall.

22. The prosthesis of clam 17, wherein the adluminal surface comprises endothelial cell-promoting structural features.

23. The prosthesis of claim 17, wherein the adluminal surface comprises endothelial cell-promoting molecules.

24. The prosthesis of claim 17, wherein the prosthesis comprises an abluminal layer that presents the adhesive abluminal surface.

25. The prosthesis of claim 17, wherein the prosthesis comprises an adluminal layer that presents the endothelial cell-promoting adluminal surface.
